(19) United States
(12) Patent Application Publication
(10) Pub. No.: US 2022/0148334 A1
(43) Pub. Date: May 12, 2022

(54) BREATHING DETECTION APPARATUS AND METHODS FOR DETECTING BREATHING

(71) Applicant: Binatone Electronics International Limited, Sheung Wan (CN)

(72) Inventor: Stephen TIN, Sheung Wan (CN)

(21) Appl. No.: 17/434,386

(22) PCT Filed: Oct. 14, 2020
(86) PCT No.: PCT/IB2020/059664

§ 371 Date: Aug. 26, 2021

(30) Foreign Application Priority Data
Oct. 14, 2019 (GB) 1914842.8

(51) Int. Cl. G06V 40/20; H04N 5/232; A61B 5/11; A61B 5/08; A61B 5/113

(52) U.S. Cl. CPC G06V 40/20; H04N 5/23216; A61B 2503/04; A61B 5/0816; A61B 5/113; A61B 5/1128

(57) ABSTRACT

A breathing detection apparatus has a first camera (12), a second camera (14) and a processing device (16). The processing device (16) is configured to perform breathing detection analysis using data relating to respective images captured by the first and second cameras (12, 14) to detect breathing characteristics of a person shown in the images.

### FIELD OF THE INVENTION

The invention relates to breathing detection apparatus and methods for detecting breathing

### BACKGROUND TO THE INVENTION

It is known to use a still or moving image camera to monitor the breathing of a child. For example, a camera may be installed in nursery and image data used to monitor the breathing of a baby or child occupying the nursery. Known methods make use of one camera. This limits the effectiveness of the breathing detection since the image of the monitored subject, is acquired from one angle only.

### SUMMARY OF THE INVENTION

The invention provides breathing detection apparatus as specified in claim 1.

The invention also includes infant monitoring apparatus as specified in claim 7.

The invention also includes a method of detecting breathing as specified in claim 8.

### BRIEF DESCRIPTION OF THE DRAWINGS

In order that the invention may be well understood, some examples thereof, which are given by way of example only, will now be described with reference to the drawings in which:

FIG. 1 is a schematic illustration of a breathing apparatus in use in an infant monitoring apparatus; and

FIG. 2 is a schematic illustration of the breathing detection apparatus of FIG. 1 in one possible use configuration.

### DETAILED DESCRIPTION

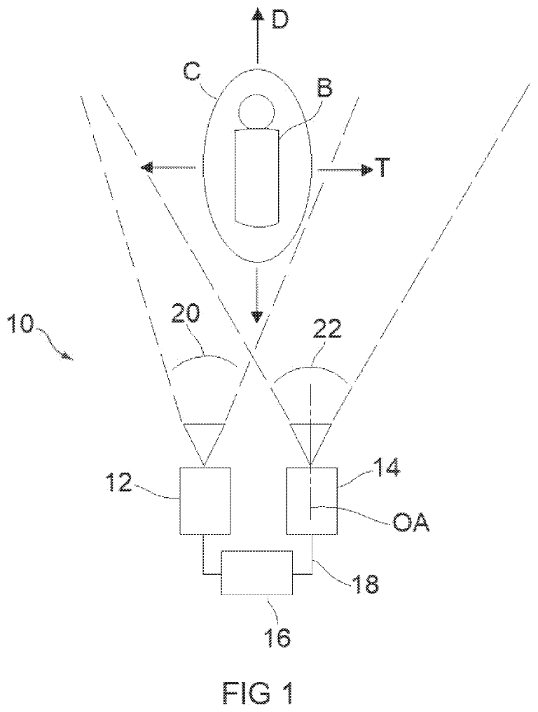

Referring to FIG. 1, a breathing detection apparatus 10 comprises a first camera 12, a second camera 14 and a processing device 16. The first and second cameras may be still or video cameras. The processing device 16 is connected with the first and second cameras to receive image data from the cameras. The connection may be a hard-wired connection 18. Alternatively, the first and second cameras 12, 14 may be provided with wireless transmitters configured to transmit the image data to wireless receiver associated with the processing device 16. The wireless receiver may be a part of the processing device or a separate device connected with the processing device. In some examples, the first and second cameras 12, 14 and processing device 16 may be provided with respective transceivers so that image data can be transmitted wirelessly to the processing device and commands can be transmitted wirelessly from the processing device to at least one of the first and second cameras.

The first and second cameras 12, 14 may be located in, for example, a nursery to capture images of a baby B in a crib C. The processing device 16 may be located in the same room or another room altogether. The breathing detection apparatus 10 may be configured as a part of an infant monitoring apparatus. The infant monitoring apparatus may take any known form and may comprise components and systems for collecting data concerning the baby B, such as body temperature, causing alerts to be notified to specified users and controlling the environment in the bedroom, for example, by controlling light levels, the room temperature or playing soothing music or other sounds such as recorded messages from a parent.

The first and second cameras 12, 14 may be configured such that the first camera 12 has a first viewing angle 20 and the second camera 14 has a second viewing angle 22 that is greater than the first viewing angle. The images captured by the first camera 12 having a relatively narrow viewing angle may be more useful for analysing breath detection indicators in the direction T transverse to the optical axis OA of the first and second cameras 12,14 and images captured by the second camera 14 having a relatively wide viewing angle may be more useful for analysing breath detection indicators in the direction D of the optical axis OA of the second camera.

The processing device 16 may be configured to set the aperture setting of at least one of the first and second cameras 12, 14. The processing device 16 may be configured to set the image capturing rate of at least one of the first and second cameras 12, 14. The processing device 16 may be configured to perform a breathing detection analysis based on at least one of:
i) the aperture settings of the first and second cameras;
ii) the frame rate of the first and second cameras; and
iii) the respective viewing angles of said first and second cameras.

The breathing detection apparatus 10 may be used in a breathing detection method comprising: i) receiving first image data from the first camera 12; ii) receiving second image data from the second camera 14; and iii) performing breathing detection analysis using the first image data and the second image data to detect at least one breathing characteristic of the baby B. The breathing detection analysis may use at least one of: i) the aperture settings of said first and second cameras; ii) the frame rate of said first and second cameras; and iii) the respective viewing angles 20, 22 of said first and second cameras. Where the respective viewing angles are used, the distance between the respective optical axes OA of the first. and second cameras 12, 14.

In examples in which the first and second cameras 12, 14 are video cameras, the first and second image data may comprise respective periodic characteristics of the respective images captured by said first and second cameras and a breathing detection analysis may comprise determining the breathing rate of the baby B using the said respective periodic characteristics.

It is to be understood that while the breathing detection apparatus 10 is particularly suitable for monitoring the breathing of a baby, or infant, it is not limited to such use and may, for example, be used for monitoring patients or elderly people in, for example, care homes.

The breathing analysis may provide measurements of breathing pattern and breathing rate.

The processing device may be a dedicated processor of a monitoring system and may comprise circuitry configured to generate and transmit commands to at least one of the first and second cameras. In some examples, the processing device may comprise a computer loaded with suitable software configured to enable the computer to communicate with the first and second cameras and, in particular, to receive image data from the cameras.

It is to be understood that although the example illustrated by FIG. 1 has first and second cameras 12, 14 with different viewing angles 20, 22, this is not essential and, in some examples, the two cameras may have the same viewing angles.

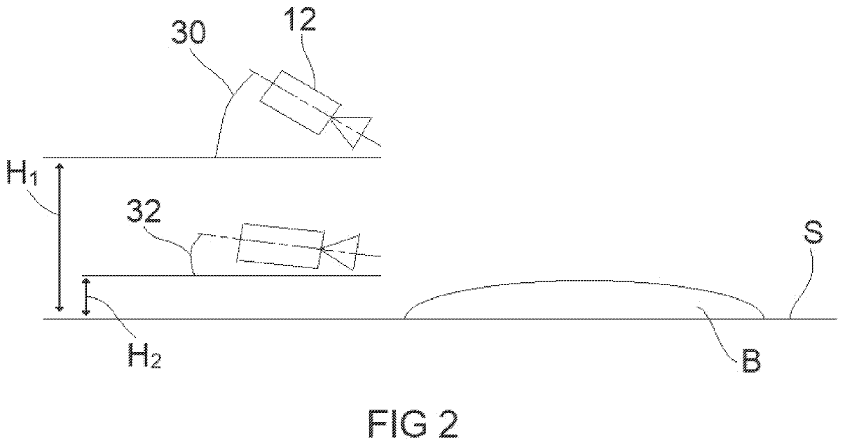

Referring to FIG. 2, in one operational configuration, the first and second cameras 12, 14 may be located at different heights with respect to the target area in which the baby B is located. For example, the first camera 12 may be located at a height Hi above the surface S on which the baby B is located and the second camera 14 may be located at a height H₂ that, is less than the height H₁. In order to point at the target area, the first camera 12 is inclined at an angle 30 to the surface S and the second camera 14 may be inclined at an angle 32 that is less than the angle 30. The relatively greater inclination of the first camera 12 to the baby B, or surface S, will make it more effective than the second camera in detecting movement in the D and T directions. The relatively lower inclination of the second camera 14 will make it more effective than the first camera 12 in detecting movement in the Z direction.

Using two cameras to provide data for breathing detection apparatus and breathing analysis processes provides the possibility of more accurate breathing detection and monitoring than conventional apparatus that uses just one camera.

The breathing detection apparatus and methods for detecting breathing disclosed herein may be used in at least in part in combination with the dual video camera infant monitoring apparatus and methods disclosed in the Applicant's United Kingdom Patent Application Number 194855.0 filed on 14 Oct. 2019 and PCT Patent Application filed 14 Oct. 2020 with the Agent's Reference P00649WO, the entire content of which is incorporated by reference.

1. A breathing detection apparatus comprising:
a first camera;
a second camera; and
a processing device configured to perform breathing detection analysis using data relating to respective images captured by said first and second cameras to detect breathing characteristics of a person shown in said images.

2. The breathing detection apparatus as claimed in claim 1, wherein at least one of said first and second cameras is a video camera.

3. The breathing detection apparatus as claimed in claim 1, wherein said processing device is configured to set the aperture setting of at least one of said first and second cameras.

4. The breathing detection apparatus as claimed in claim 1, wherein said processing device is configured to set the image capturing rate of at least one of said first and second cameras.

5. The breathing detection apparatus as claimed in claim 1, wherein said first camera to configured to have a first viewing angle and said second camera is configured to have a second viewing angle and said second viewing angle is greater than said first viewing angle.

6. The breathing detection apparatus as claimed in claim 1, wherein said processing device is configured to perform said breathing detection analysis at least in part based on at least one of:
i) the aperture settings of said first and second cameras;
ii) the frame rate of said first and second cameras; and
iii) the respective viewing angles of said first and second cameras.

7. An infant monitoring apparatus comprising a breathing detection apparatus, comprising:
a first camera;
a second camera; and
a processing device configured to perform breathing detection analysis using data relating to respective images captured by said first and second cameras to detect breathing characteristics of a person shown in said images.

8. A method of detecting breathing comprising:
receiving first image data from a first camera arranged to capture images of a person;
receiving second image data from a second camera arranged to capture images of said person; and
performing breathing detection analysis using said first image data and said second image data to detect at least one breathing characteristic of said person.

9. The method of detecting breathing as claimed in claim 8, wherein performing said breathing detection analysis uses at least one of:
i) the aperture settings of said first and second cameras;
ii) the frame rate of said first and second cameras; and
iii) the respective viewing angles of said first and second cameras.

10. The method as claimed in claim 8, wherein:
said first and second cameras are video cameras;
said first and second image data comprises respective periodic characteristics of the respective images captured by said first and second cameras; and
performing said breathing detection analysis comprises determining breathing rate using the said respective periodic characteristics.

11. The infant monitoring apparatus as claimed in claim 7, wherein at least one of said first and second cameras is a video camera.

12. The infant monitoring apparatus as claimed in claim 7, wherein said processing device is configured to set the aperture setting of at least one of said first and second cameras.

13. The infant monitoring apparatus as claimed in claim 7, wherein said processing device is configured to set the image capturing rate of at least one of said first and second cameras.

14. The infant monitoring apparatus as claimed in claim 7, wherein said first camera to configured to have a first viewing angle and said second camera is configured to have a second viewing angle and said second viewing angle is greater than said first viewing angle.

15. The infant monitoring apparatus as claimed in claim 7, wherein said processing device is configured to perform said breathing detection analysis at least in part based on at least one of:
i) the aperture settings of said first and second cameras;
ii) the frame rate of said first and second cameras; and
iii) the respective viewing angles of said first and second cameras.
